(19) United States
(12) Patent Application Publication
(10) Pub. No.: US 2025/0312086 A1
(43) Pub. Date: Oct. 9, 2025

(54) MEDICAL DEVICES, SYSTEMS, AND METHODS FOR PROVIDING TRACTION TO TISSUE

(71) Applicant: Boston Scientific Scimed, Inc., Maple Grove, MN (US)

(72) Inventors: Mauro VIQUEZ GONZALEZ, San Isidro (CR); Oscar Andres SEGURA ELIZONDO, San Jose (CR); Gonzalo Jose SAENZ VILLALOBOS, Alajuela (CR); Marco Vinicio ARIAS ALPIZAR, San José (CR); Fernando VALERIO GUERRERO, Poas (CR); Viviana ARTAVIA SALAS, Ulloa (CR); Erick Gerardo VAN PATTEN RIVERA, San Jose (CR); Gustavo CARVAJAL BONILLA, San Joaquín (CR); Valeria ZUÑIGA VARGAS, Austin, TX (US); Rafael BERENZON, Cartago (CR); Gabriela GAMBOA BASTOS, San Jose (CR); Esteban de la Trinidad JIMENEZ SANCHEZ, San Jose (CR)

(73) Assignee: Boston Scientific Scimed, Inc., Maple Grove, MN (US)

(21) Appl. No.: 19/171,962

(22) Filed: Apr. 7, 2025

Related U.S. Application Data
(60) Provisional application No. 63/631,637, filed on Apr. 9, 2024.

(51) Int. Cl. A61B 18/14; A61B 17/00; A61B 17/02; A61B 18/00

(52) U.S. Cl. CPC A61B 18/1445; A61B 17/0218; A61B 2017/00269; A61B 2017/00349; A61B 2018/00601; A61B 2018/00982; A61B 2018/1455

(57) ABSTRACT

Disclosed is a medical system that may comprise: a shaft having a working channel; a first medical instrument extending through the working channel; a second medical instrument disposed radially outside the shaft. The first medical instrument may be a tensioning tool and the second medical instrument may be a cutting tool comprising a first blade and a second blade. The first blade may be attached to a first actuation wire and the second blade may be attached to a second actuation wire.

### CROSS-REFERENCE TO RELATED APPLICATION(S)

This application claims the benefit of priority to U.S. Provisional Application No. 63/631,637, filed on Apr. 9, 2024, which is incorporated by reference herein in its entirety.

### TECHNICAL FIELD

Various aspects of this disclosure generally relate to medical devices and systems and for providing traction to tissue and related methods. In particular, aspects of this disclosure relate to medical devices, systems, and related methods for devices that apply traction to tissue to facilitate resection.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. For example, an instrument, such as a surgical knife, may be used via a working channel of an endoscope in order to cut and remove the lesion. In some procedures, a lifting agent may be injected under the lesion prior to removing the lesion, so as to facilitate access by the surgical knife or other instrument. A need exists for applying traction or tension to a lesion or other portion of a body lumen to facilitate medical procedures, such as ESD.

### SUMMARY

Each of the aspects disclosed herein may include one or more of the features described in connection with any of the other disclosed aspects.

In an example, a medical system may comprise: a shaft having a working channel; a first medical instrument extending through the working channel; a second medical instrument disposed radially outside the shaft. The first medical instrument may be a tensioning tool and the second medical instrument may be a cutting tool comprising a first blade and a second blade. The first blade may be attached to a first actuation wire and the second blade may be attached to a second actuation wire.

Any of the aspects disclosed herein may include any of the following features, alone or in combination. The tensioning tool may comprise an actuation cable and a hook. The tensioning tool may comprise an actuation cable and a forceps. The first actuation wire and the second actuation wire may be controlled by a single actuator. The first actuation wire and the second actuation wire may move in concert. The first actuation wire is controlled by a first actuator and the second actuation wire is controlled by a second actuator. The first actuation wire and the second actuation wire may be disposed on opposite sides of the shaft. Each of the first blade and the second blade may comprise: a substantially straight inner edge, a substantially curved outer edge, and a hinge portion. The wire may be attached to the first blade at the hinge portion of the first blade, and the second actuation wire may be attached to the second blade at the hinge portion of the second blade. The cutting tool may comprise an open position and a closed position. In the open position, the first blade and the second blade may be separated from each other, and in the closed position, the first blade and the second blade may be brought into proximity with each other. The first blade may pivot about the hinge portion of the first blade to transition from the open position to the closed position, and the second blade may pivot about the hinge portion of the second blade to transition from the open position to the closed position. The cutting tool may comprise an electrosurgical element. The first blade may be serrated and the second blade may be serrated. A coupling portion may be provided for coupling the cutting tool to the shaft. The first blade and the second blade may be movable toward and away from each other along a track. The track may be formed on a coupling element that couples the cutting tool to the shaft.

In another aspect, a medical system may comprise: an endoscope having a shaft; an end cap disposed at a distal end of the shaft, the end cap including a through channel; a first medical instrument configured to perform a resection of a target tissue, the first medical instrument extending through a working channel of the shaft; a second medical instrument configured to apply traction to the target tissue, the second medical instrument including an actuation cable extending through the through channel. The actuation cable may comprise a proximal portion that is parallel to the shaft, and a distal portion that is disposed at an angle to the shaft.

Any of the aspects disclosed herein may include any of the following features, alone or in combination. The through channel may include a bend.

In another aspect, a medical method may comprise: inserting an endoscope into a body lumen of a subject, the endoscope having: a shaft and an end cap disposed at a distal end of the shaft, the end cap including a through channel, wherein: a first medical instrument includes an actuation cable extending through the through channel; a second medical instrument extends through a working channel of the shaft; advancing the endoscope through the body lumen to a target tissue; actuating the first medical instrument to apply traction to the target tissue; actuating the second medical instrument to perform a resection of the target tissue.

Any of the aspects disclosed herein may include any of the following features, alone or in combination. The actuation cable may comprise a proximal portion that is parallel to the shaft, and a distal portion that is disposed at an angle to the shaft. The through channel may include a bend.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts an exemplary system for applying traction and performing resection of a tissue.

FIG. 2 depicts a top view of the exemplary system of FIG. 1 in an exemplary use.

FIG. 3 depicts aspects of another exemplary system for applying traction and performing resection of a tissue.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. In some of the drawings, arrows labeled “P” and “D” label proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Disclosed are systems, devices, and methods for applying traction to tissue. During an endoscopic procedure, one or more accessory devices may be used along with a medical device (e.g., an endoscope) in order to perform a medical procedure such, as, for example, tissue resection. In order to facilitate resection, the disclosed systems and/or devices may apply traction to a target tissue to be resected. The traction may facilitate tissue resection by pulling the target tissue away from adjacent walls of a body lumen. As the target tissue is being resected, the disclosed systems and/or devices may continue to apply traction to the target tissue, to lift away the tissue and move it out of the way to provide for increased visualization using a camera of the medical device.

Although the term endoscope may be used herein, it will be appreciated that the disclosure encompasses systems other devices or accessories for other devices, including, but not limited to, duodenoscopes, colonoscopes, ureteroscopes, bronchoscopes, laparoscopes, cytoscopes, hysteroscopes, sheaths, catheters, tomes, or any other delivery device.

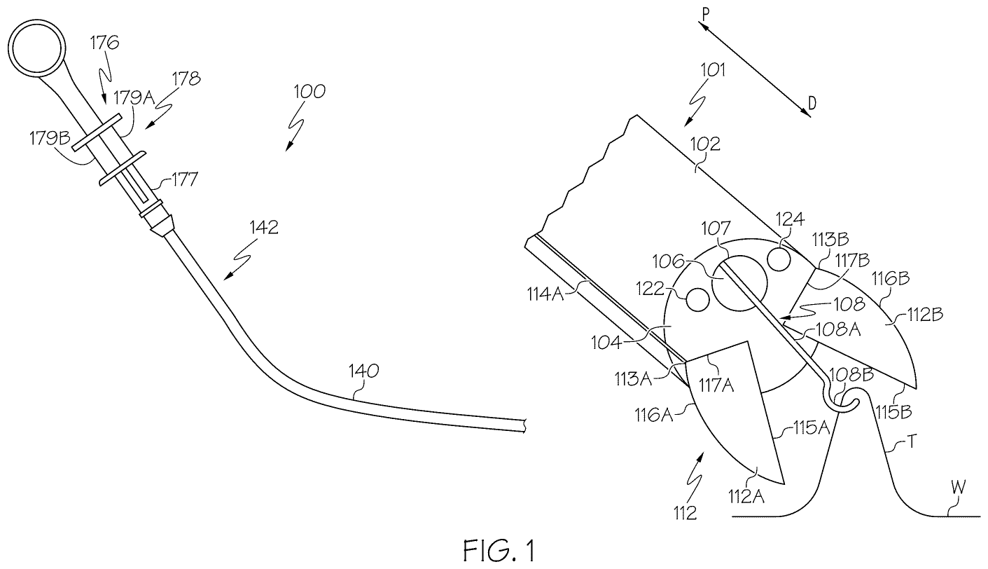

FIG. 1 depicts an exemplary medical device 101 (e.g., an endoscope or other type of scope device or insertion device) which may be a part of a larger medical system 100. The medical device 101 may include a sheath or shaft 102 and a distal tip 104. The distal tip 104 may be a distalmost face of shaft 102 and may include an imaging device 122 (e.g., a camera) and a lighting source 124 (e.g., an LED or an optical fiber). Distal tip 104 may be forward-facing. That is, the imaging device 122 and the lighting source 124 may face distally (e.g., approximately parallel to a longitudinal axis of shaft 102 and distal tip 104). Alternatively, the distal tip 104 may be side-facing (the imaging device 122 and/or the lighting source 124 may face radially outward) or may include a combination of side-facing and forward-facing features. In use, an operator may insert at least a portion of the shaft 102 into a body lumen of a subject. The distal tip 104 of the shaft 102 may be navigated to a procedure site in the body lumen to perform a medical procedure.

The system 100 may also include a first accessory device 108, which may be a hook (e.g., a traction hook). The first accessory device 108 may be delivered through the shaft 102 of the medical device 101 via a working channel 106 to the distal tip 104. The first accessory device 108 may exit the working channel 106 at an opening 107 on the distal tip 104. The accessory device 108 may comprise an actuation portion 108A (e.g, a wire, rod, or cable), and a hook portion 108B, wherein the hook portion 108B is configured and shaped to grasp tissue in a body lumen, such as a target tissue T. The first medical instrument may, alternatively, include a forceps device, a clip device, a barb, a snare, a forceps, or any other device capable of and configured to apply traction to tissue.

A second accessory device 112, such as a surgical cutting tool, may be installed over the shaft 102 to the distal tip 104 via an over-the-scope arrangement. For example, the second accessory device 112 may be fitted over the distal tip 104 before the distal tip 104 is inserted into a body lumen of a subject. In some examples, the second accessory device 112 may include a cap, a band, or another element for affixing the second accessory device 112 on an outer/external surface of the shaft 102.

In some examples, the second accessory device 112 may comprise two blades, a blade 112A and a blade 112B. The blade 112A may be attached to an actuation wire 114A. Although the term “wire” is used herein, it will be appreciated that the wire 114A may alternatively be or include a cable, a rod, or any other actuation member. The blade 112A may be pivotable or rotatable about a hinge 113A. For example, the hinge 113A may couple the blade 112A to a coupling element that couples the second accessory device 112 to the shaft 102, directly to the shaft 102, and/or to the wire 114A. Similarly, the blade 112B may be attached to an actuation wire 114B. The blade 112B may be pivotable or rotatable about a hinge 113B. For example, the hinge 113B may couple the blade 112B to a coupling element that couples the second accessory device 112 to the shaft 102, directly to the shaft 102, and/or to the wire 114B. The wires 114A and 114B may extend longitudinally along an outer/exterior surface of the shaft 102. Alternatively, the blades 112A and 112B may be movable toward and away from each other along a track (e.g., a track formed on a coupling element that couples the second accessory device to the shaft 102).

Blade 112A may have any desired shape. In the example shown in FIG. 1, blade 112A has a substantially straight inner/inside cutting edge 115A arranged medially (toward a central longitudinal axis of shaft 102 and toward blade 112B), a curved outer/outside edge 116A, and a substantially straight proximal edge 117A. The proximal edge 117A may be substantially perpendicular to the cutting edge 115A. In other examples, outside edge 116A may be straight, such that blade 112A is approximately triangular. The hinge 113A may be disposed at a proximal end of blade 112A adjacent the outside edge 116A of blade 112A, near or at where outside edge 116A meets proximal edge 117A.

Blade 112A and blade 112B may be of substantially the same shape and dimension. In the example shown in FIG. 1, blade 112B, like blade 112A has a substantially straight cutting edge 115B arranged medially, a curved outside edge 116B, and a substantially straight proximal edge 117B. The proximal edge 117B may be substantially perpendicular to the cutting edge 115B. Outside edge 116B may also be straight, such that blade 112B is approximately triangular. The hinge 113B, similar to hinge 113A, may be disposed at a proximal end of blade 112B adjacent the outside edge 116B of blade 112B, near or at where outside edge 116B meets proximal edge 117B.

In use, first accessory device 108 may apply traction and/or tension to the target tissue T (e.g., to a lesion or to an area neighboring a lesion), pulling target tissue T away from a surrounding (e.g., adjacent) tissue wall W. The second medical instrument, e.g., second accessory device 112, may resect target tissue T.

A handle 176 may be disposed at a proximal end of the first accessory device 108 and/or second accessory device 112. In some examples, as shown, the same handle 176 may be used to control the first accessory device 108 and the second accessory device. Alternatively, separate handles 176 may be used to control first accessory device 108 and second accessory device 112. The handle 176 may include a handle body 177 and one or more actuators 178. The actuator(s) 178 may be used for controlling aspects of the first accessory device 108 and the second accessory device 112. For example, the actuator 178 may be a spool that is longitudinally movable along the handle body 177. The handle 176 may have any of the features of any medical device handle known in the art. The handle 176 may include additional and/or alternative actuators. A configuration of the handle 176 is merely exemplary and shown for illustrative purposes.

In an embodiment, the actuator 178 may be divided into two distinct actuators or spools, a first actuator 179A and a second actuator 179B. The first actuator 179A and the second actuator 179B may actuate separately. For example, first actuator 179A may actuate hook portion 108B and second actuator 179B may actuate second accessory device 112.

For example, the first actuator 179A may be coupled to the actuation portion 108A. In an embodiment, sliding the first actuator 179A distally may actuate the hook portion 108B to move distally via actuating portion 108A, and sliding the first actuator 179A proximally may actuate the hook portion 108B to move proximally.

The second actuator 179B may be coupled to the actuation wires 114A, 114B. Sliding the second actuator 179B distally may actuate the second accessory device 112 (e.g., close or open the blades 112A and 112B), and sliding the second actuator 179B proximally may actuate the surgical cutting tool (e.g., open or close the blades 112A and 112B). Although sliding of actuators 179A, 179B is referred to above, it will be appreciated that actuators 179A, 179B may be actuated by alternative movements, depending on a type of actuator used.

In some examples, proximal portions of actuating portion 108A and actuation wires 114A, 114B may be disposed in a shaft 140 of an insertion portion 142 that extends distally from the handle 176. In other examples, the shaft 140 may be omitted.

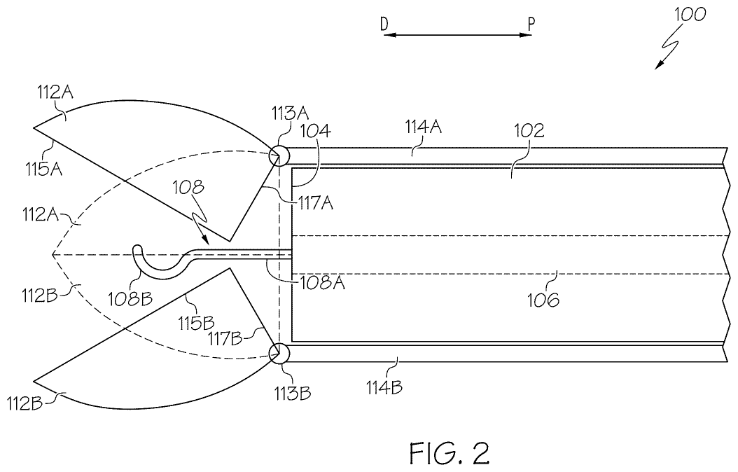

FIG. 2 depicts a top view of the exemplary system of FIG. 1 in an exemplary use. As depicted in FIG. 2, the second accessory device 112 may have an open position, shown in solid lines, and a closed position, shown in dashed lines. In the open position, the cutting edge 115A of blade 112A and the cutting edge 115B of blade 112B are separated from each other, and the proximal edge 117A of blade 112A and the proximal edge 117B are lifted away (separated) at their medial ends from distal tip 104 (e.g., from a distal face of distal tip 104). In the closed position, the cutting edge 115A of blade 112A is in contact with or in close proximity to the cutting edge 115B of blade 112B. The proximal edges 117A and 117B may be approximately parallel to distal tip 104. The second accessory device 112 may be configured such that the cutting edge 115A of blade 112A and the cutting edge 115B of blade 112B interact to effectively cut the tissue T shown in FIG. 1 when the second accessory device 112 is transitioned to the closed position. In aspects, an electrosurgical element may be coupled to or incorporated into blades 112A, 112B of the second accessory device 112. For example, a wire may carry electrical energy to one or more of blades 112A, 112B, which may be conductive. In additional or alternative aspects, each of cutting edge 115A and cutting edge 115B may include one or more serrated portions. In aspects, blades 112A, 112B may be configured such that the cutting edge 115A and cutting edge 115B overlap in the closed position, etc.

The second accessory device 112 may be transitioned between the open position and the closed position by actuation of each of actuation wire 114A of blade 112A and actuation wire 114B of each of blade 112B. Actuation wire 114A may be attached to blade 112A and actuation wire 114B may be attached to blade 112B. Each of the wires 114A and 114B may be coupled to an actuator 132 of a handle 130. In some examples, both of the wires 114A and 114B may be coupled to the same actuator 132. In alternatives, the wires 114A and 114B may be coupled to different actuators. The actuator 132 may be movable to move the wires 114A, 114B proximally or distally in order to actuate the blades 112A, 112B. For example, movement of the wires 114A and 114B distally (or proximally) may cause the blades 112A, 112B to close, such that the cutting edges 115A, 115B move toward each other (e.g., toward a central longitudinal axis of shaft 102). In other words, blades 112A and 112B may be brought into proximity with each other. Movement of the wires 114A and 114B in the opposite direction may cause the blades 112A, 112B to open, such that cutting edges 115A, 115B move away from one another (e.g., away from the central longitudinal axis of the shaft 102) so that they are separated from each other.

For example, when the actuation wire 114A is moved in a distal direction, blade 112A may pivot around hinge 113A to transition the blade 112A from the open position to the closed position. When the actuation wire 114A is moved in the distal direction, actuation wire 114B may be moved in the distal direction as well in concert with the actuation wire 114A. As such, when blade 112A is moved from the open position to the closed position, blade 112B may also be moved (transitioned) from the open position to the closed position.

Similarly, when the actuation wire 114A is moved in a proximal direction opposite to the distal direction, blade 112A may pivot around hinge 113A to transition the blade 112A from the closed position to the open position. When the actuation wire 114A is moved in the proximal direction, actuation wire 114B may be moved in the proximal direction as well in concert with the actuation wire 114A. As such, when blade 112A is moved from the closed position to the open position, blade 112B may also moved (transitioned) from the closed position to the open position.

An operator may insert the medical device 101 into a body lumen of a subject, and advance the shaft 102 of the medical device 101 through the body lumen of the subject toward the target tissue T. The target tissue T may be a lesion or ulcer, or other tissue requiring resection or treatment. The target tissue T may be disposed on the tissue wall W of the body lumen. The imaging device 122 (e.g., camera) and the lighting source 124 (e.g., an LED or optical fiber) may be used to guide the shaft 102 to the target tissue T. When medical device 101 is inserted into the body lumen, the blades 112A, 112B may be in a closed configuration to avoid tissue damage.

When the distal tip 104 of the shaft 102 has arrived at the target tissue T, the operator may actuate the first accessory device 108 to apply traction to the target tissue T, pulling target tissue T away from a surrounding (e.g., adjacent) tissue wall W. The hook portion 108B of first accessory device 108 may be moved proximally or distally via actuation of actuation portion 108A of first accessory device 108 to ensure adequate traction is applied to the target tissue T. As discussed above, the actuation portion 108A may be an actuation wire or actuation cable that is controlled by actuator 179A on handle 176 controlled by the operator. The operator may use the imaging device 122 (e.g., camera) and the lighting source 124 (e.g., LED or optical fiber) to determine if adequate traction has been applied to the target tissue T.

Prior to or after applying traction with first accessory device 108, the operator may open blades 112A and 112B (e.g., by pulling actuation wires 114A and 114B proximally, together or separately). The operator may use the imaging device 122 (e.g., camera) and the lighting source 124 (e.g., LED or optical fiber) to position the second accessory device 112 to perform a resection of the target issue T. The operator may then actuate the actuation wire 114A and the actuation wire 114B to bring the cutting edge 115A of blade 112A and the cutting edge 115B of blade 112B together, thereby cutting the target tissue T away from tissue wall W. The actuation wire 114A and the actuation wire 114B, as described above, may be actuated in concert so that they move proximally and distally together so that the second accessory device 112 may be transitioned between an open position and a closed position by a single actuator, e.g., actuator 179B.

As discussed above, the actuation wire 114A and the actuation wire 114B may be disposed outside the shaft 102 of the medical device 101 in an over-the-scope arrangement, while the actuation portion 108A of the first accessory device 108 is disposed in the working channel 106 in the shaft 102 of the medical device 101. In some examples, first accessory device 108 may be moved proximally through working channel 106 after the target tissue T is resected, with first accessory device 108 still coupled to the target tissue T. Thus, first accessory device 108 may be used in order to collect target tissue T for further analysis (e.g., pathology). In other examples, first accessory device 108 may be removed from working channel 106 after first accessory device 108 is uncoupled from the target tissue T, and another accessory device (e.g., a basket) may be inserted into working channel 106 in order to collect and remove the target tissue T.

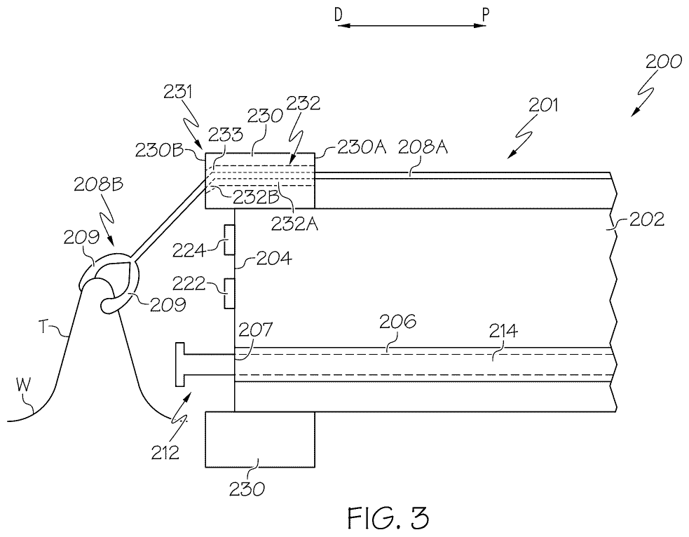

FIG. 3 depicts another exemplary medical device 201 (e.g., an endoscope) which may be a part of a larger medical system 200 (e.g., an endoscope system). FIG. 3 depicts a side view of a medical device 201 in a body lumen.

Medical device 201 is substantially similar to medical device 101 described above and may have any of the properties of medical device 101. Medical device 201 may likewise be an endoscope including a shaft 202, with a working channel 206 disposed in the shaft 202, the working channel 206 including an opening 207 at a distal tip 204 of the shaft 202. The distal tip 204 may include an imaging device 222 (e.g., a camera) and a lighting source 224 (e.g., an LED or an optical fiber).

An accessory device 231 may be coupled to a distal end of medical device 201. Accessory device 231 may include a cap 230 (e.g., an end cap) and an accessory device 208. The cap 230 may be disposed around the distal tip 204 of the shaft 202. The cap 230 may be an annular ring disposed around a circumferential distal edge of the distal tip 204 and/or a distal edge of the shaft 202. In alternatives, the cap 230 may also cover a portion of the distal face of the distal tip 204. A distalmost end of the cap 230 may be flush with a distalmost face of the distal tip 204, or may protrude distally from a distalmost face of the distal tip 204, as shown in FIG. 3. The cap 230 may include a through channel 232 that extends through the cap 230 from a proximal surface 230A of the end cap 230 to the distal surface 230B of the end cap 230.

The through channel 232 may have a bend 233, separating the through channel 232 into a proximal portion 232A that is parallel approximately to a central longitudinal axis of the shaft 202, and a distal portion 232B that is offset at a non-zero angle from a central longitudinal axis of the shaft 202. A longitudinal axis of the distal portion 232B may extend radially inward, toward the central longitudinal axis of the shaft 202, moving in a distal direction. A first medical instrument, such as an accessory device 208, may extend along an outer/external surface of the shaft 202 to the cap 230 via an over-the-scope arrangement. The accessory device 208 may include an end effector 208B (e.g., a forceps, grasper, hook, barb, snare, clip, etc.) and an actuation wire or cable 208A connected to the end effector 208B and configured to be actuated by the operator using a handle having any of the properties of the handles discussed above. The actuation cable 208A may pass through the through channel 232 formed in the cap 230. The bend 233 causes the distal portion 232B to direct a distal portion of the actuation cable 208A toward a central longitudinal axis of the shaft 202 as the actuation cable 208A extends in a distal direction away from the end cap 230.

A handle (e.g., having any of the properties of handle 176 depicted in FIG. 1) may be disposed at a proximal end of accessory device 208. In an embodiment, first actuator 179A may actuate end effector 208B (e.g., open and/or close the jaws 209 of end effector 208B) and the second actuator 179B may move end effector 208B proximally and/or distally in order to extend end effector 208B toward tissue and/or apply traction to the tissue by moving end effector 208B proximally.

In use, an operator may insert medical device 201 (e.g. an endoscope) into a body lumen of a subject, and advance the shaft 202 of the endoscope through the body lumen of the subject toward the target tissue T. The target tissue T may be a lesion or ulcer, or other tissue requiring resection or treatment. The target tissue T may be disposed on the tissue wall W of the body lumen. The imaging device 222 (e.g., camera) and the lighting source 224 (e.g., LED or optical fiber) may be used to guide the shaft 202 to the target tissue T.

When the distal tip 204 of the shaft 202 has arrived at the target tissue T, the operator may actuate the end effector 208B (e.g., using the handle 176) to apply traction to the target tissue T, pulling target tissue T away from a surrounding (e.g., adjacent) tissue wall W. The end effector 208B may be moved proximally or distally via proximal/distal movement of the accessory device 208. For example, the actuation cable 208A may extend through a sheath (similar to sheaths including actuation wires in medical instruments known in the art), and the sheath may be longitudinally fixed with respect to the end effector 208B, such that proximal/distal movement of the sheath causes proximal/distal movement of the end effector 208B. Alternatively, an actuation wire may be coupled to an actuator of the handle 176, and the actuation wire may be coupled to an actuator 178 of the handle 176, to cause proximal and/or distal movement of the end effector 208B.

End effector 208B may initially be moved distally toward the target tissue T. In some examples, channel 232 may be disposed on one side of a plane that extends along a central longitudinal axis of the shaft 202 and bisects the shaft. As the end effector 208B is moved distally, the end effector 208B may also cross over/through the plane, due to an angle of distal portion 232B. Thus, the end effector 208B may extend toward the traction tissue B. For example, distal tip 204 may be positioned so that the channel 232 and the target tissue T are on opposite sides of the shaft 202. Due to the angle of distal portion 232B, the end effector 208B may extend across the distal tip 204 toward the target tissue T.

Actuation of actuation cable 208A may open/close jaws 209 to grasp onto the target tissue T. For example, an actuator 178 of the handle 176 may be actuated to cause the jaws 209 to open and/or close the jaws 209. The end effector 208B may be moved proximally when grasping the target tissue T to ensure adequate traction is applied to the target tissue T. The target tissue T may be pulled away from surrounding portions of a luminal wall W. As discussed above, the actuation cable 208A may be an actuation wire or actuation cable that is controlled by an actuator on a handle controlled by the operator. The operator may use the imaging device 222 (e.g., camera) and the lighting source 224 (e.g., LED or optical fiber) to determine if adequate traction has been applied to the target tissue T.

The operator may pass a second medical device 212, such as a cutting tool (e.g., a knife), through the shaft 202 via the working channel 206 to the distal tip 204. The second medical device 212 may exit the working channel 206 at distal tip 204. The second medical device 212 may include an actuation wire 214 that passes through the working channel 206 and that is configured to be actuated by the operator to move the second medical device 212 proximally and distally. The operator may use the imaging device 222 (e.g., camera) and the lighting source 224 (e.g., LED or optical fiber) to position the second medical device 212. The second medical device 212 may be, for example, an electrosurgical knife. The second medical instrument, e.g., surgical second medical device 212, may be used to resect the target tissue T.

During resection of the target tissue T, the end effector 208B may continue to grasp and apply traction to the target tissue T, forming a tissue flap and keeping the target tissue positioned to facilitate resection. Following resection, the actuation cable 208A may be actuated so that the jaws 209 of the end effector 208B are opened, thereby releasing the target tissue T. Another medical instrument (e.g., a basket) may be used to retrieve the target tissue T and to remove the target tissue T from the subject's body.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical system comprising:
a shaft having a working channel;
a first medical instrument extending through the working channel;
a second medical instrument disposed radially outside the shaft;
wherein the first medical instrument is a tensioning tool, and the second medical instrument is a cutting tool comprising a first blade and a second blade, wherein the first blade is attached to a first actuation wire and the second blade is attached to a second actuation wire.

2. The medical system of claim 1, wherein the tensioning tool comprises an actuation cable and a hook.

3. The medical system of claim 1, wherein the tensioning tool comprises an actuation cable and a forceps.

4. The medical system of claim 1, wherein the first actuation wire and the second actuation wire are controlled by a single actuator.

5. The medical system of claim 1, wherein the first actuation wire and the second actuation wire move in concert.

6. The medical system of claim 1, wherein the first actuation wire is controlled by a first actuator and the second actuation wire is controlled by a second actuator.

7. The medical system of claim 1, wherein the first actuation wire and the second actuation wire are disposed on opposite sides of the shaft.

8. The medical system of claim 1, wherein each of the first blade and the second blade comprises:
a substantially straight inner edge,
a substantially curved outer edge, and
a hinge portion; wherein:
the first actuation wire is attached to the first blade at the hinge portion of the first blade, and the second actuation wire is attached to the second blade at the hinge portion of the second blade.

9. The medical system of claim 8, wherein the cutting tool comprises an open position and a closed position, wherein:
in the open position, the first blade and the second blade are separated from each other, and in the closed position, the first blade and the second blade are brought into proximity with each other.

10. The medical system of claim 9, wherein the first blade pivots about the hinge portion of the first blade to transition from the open position to the closed position, and the second blade pivots about the hinge portion of the second blade to transition from the open position to the closed position.

11. The medical system of claim 1, wherein the cutting tool comprises an electrosurgical element.

12. The medical system of claim 1, wherein the first blade is serrated and the second blade is serrated.

13. The medical system of claim 1, further comprising a coupling portion for coupling the cutting tool to the shaft.

14. The medical system of claim 1, wherein the first blade and the second blade are movable toward and away from each other along a track.

15. The medical system of claim 14, wherein the track is formed on a coupling element that couples the cutting tool to the shaft.

16. A medical system comprising:
an endoscope having a shaft;
an end cap disposed at a distal end of the shaft, the end cap including a through channel;
a first medical instrument configured to perform a resection of a target tissue, the first medical instrument extending through a working channel of the shaft;
a second medical instrument configured to apply traction to the target tissue, the second medical instrument including an actuation cable extending through the through channel; wherein,
the actuation cable comprises a proximal portion that is parallel to the shaft, and a distal portion that is disposed at an angle to the shaft.

17. The medical system of claim 16, wherein the through channel includes a bend.

18. A medical method comprising:
inserting an endoscope into a body lumen of a subject, the endoscope having:
a shaft and an end cap disposed at a distal end of the shaft, the end cap including a through channel, wherein:
a first medical instrument includes an actuation cable extending through the through channel;
a second medical instrument extends through a working channel of the shaft;
advancing the endoscope through the body lumen to a target tissue;
actuating the first medical instrument to apply traction to the target tissue;
actuating the second medical instrument to perform a resection of the target tissue.

19. The medical method of claim 18, wherein the actuation cable comprises a proximal portion that is parallel to the shaft, and a distal portion that is disposed at an angle to the shaft.

20. The medical method of claim 18, wherein the through channel includes a bend.
